(19) United States
(12) Patent Application Publication
(10) Pub. No.: US 2026/0363774 A1
(43) Pub. Date: Nov. 19, 2026

(54) METHODS FOR KINEMATIC ALIGNMENT IN NAVIGATED TOTAL KNEE ARTHROPLASTY (TKA)

(71) Applicant: GLOBUS MEDICAL, INC., AUDUBON, PA (US)

(72) Inventors: Olivier Chappuis, Lutry (CH); David Stumpo, Collegeville, PA (US); Loris Duch, Bretigny-sur-Morrens (CH)

(21) Appl. No.: 19/210,293

(22) Filed: May 16, 2025

(51) Int. Cl. A61B 34/10; A61B 17/15; A61B 17/17; A61B 17/56; A61B 34/20

(52) U.S. Cl. CPC A61B 34/10; A61B 17/157; A61B 17/1703; A61B 2017/564; A61B 2034/105; A61B 2034/107; A61B 2034/2055

(57) ABSTRACT

Methods, systems, and devices are provided for performing a total knee arthroplasty (TKA) procedure to provide a patient with kinematic alignment of the knee. Such methods, systems, and devices include image-based and imageless workflows for pre-operatively or intra-operatively identifying the patient's native posterior tibial slope, and planning the TKA procedure based on the identified slope, including planning of the tibial cut plane.

### BACKGROUND

The disclosure relates generally to devices, systems, and methods for use in performing total knee arthroplasty (TKA) procedures. More particularly, the disclosure relates to methods, systems, and devices for planning tibial and femoral implant placement in TKA procedures to provide patient leg kinematic alignment (KA).

Kinematic alignment (KA) is a technique for TKA that aims to reconstruct patient-specific limb alignment, joint lines, and knee biomechanics based on pre-arthritic kinematic axes of the patient's leg. During TKA procedures, this is achieved by resurfacing the distal femur and proximal tibia, and by positioning appropriate femoral and tibial prostheses, such as a femoral implant, tibial tray, and tibial insert for a single knee.

A variety of philosophies may be used for aligning knee components in a TKA procedure, including constitutional alignment techniques (FIG. 1A), systematic techniques such as mechanical alignment (MA) and anatomical alignment (AA) (FIG. 1B), and kinematic alignment (KA) (FIG. 1C).

The KA technique for TKA was developed following observations that TKAs performed using systematic techniques, such as MA and AA, are often affected by residual complications. By targeting a more physiological, personalized, and reproducible implantation, the KA technique aims to improve prosthetic knee function, patient satisfaction, and component lifespan, compared to conventional techniques for knee replacement. Accordingly, solutions are needed to acquire key parameters of a patient's native alignment for use in KA-TKAs.

### BRIEF DESCRIPTION

A first aspect of the disclosure provides a method for planning a total knee arthroplasty (TKA) procedure on a patient, comprising obtaining a pre-operative image of a knee of the patient; identifying, on the pre-operative image, a reference line corresponding to a native posterior tibial slope of the patient; and planning a tibial cut plane based on the reference line.

In certain embodiments, the tibial cut plane is planned such that, when a tibia of the patient is cut along the tibial cut plane, and a tibial tray is inserted onto a prepared proximal surface of the tibia, the tibial tray provides a posterior tibial slope corresponding to the native posterior tibial slope of the patient.

In certain embodiments, the pre-operative image comprises a 3D model of a tibia of the patient generated from a CT scan or an MRI scan.

In certain embodiments, identifying the reference line comprises extracting from the 3D bone model a deepest proximal plateau point at one or both of a medial side or a lateral side of the tibia of the patient; creating a measurement plane parallel to a sagittal plane of the patient; translating the measurement plane to the deepest proximal plateau point corresponding to a side of evaluation; and extracting a first most proximal point and a second most proximal point of the 3D bone model intersecting the measurement plane.

In certain embodiments, the deepest proximal plateau point is extracted on the medial side of the tibia.

In certain embodiments, the steps of extracting the deepest proximal plateau point, and extracting first most proximal point and the second most proximal point are performed automatically using a surgical computer platform.

Certain embodiments further comprise drawing the reference line on the pre-operative image between the first most proximal point and the second most proximal point, wherein the reference line corresponds to the native posterior tibial slope.

Certain embodiments further comprise intra-operatively confirming a position of the reference line corresponding to the native tibial slope by providing a camera tracking system adapted to track a pose of one or more tracking arrays, and a computer platform in communication with the camera tracking system; affixing a first tracking array to a tibia of the patient, wherein the first tracking array comprises a plurality of reference elements adapted for tracking by the camera tracking system; registering a pose of the tibia to the computer platform; providing a navigated instrument comprising a second tracking array having a plurality of reference elements thereon and adapted for tracking by the camera tracking system; using the navigated instrument, directly acquiring an axis corresponding to a native posterior tibial slope of the tibia; and confirming the position of the reference line as identified pre-operatively.

In certain embodiments, directly acquiring the axis comprises aligning a shaft of the navigated instrument with the native posterior tibial slope; and capturing an axis of the native posterior tibial slope in response to a user input.

Certain embodiments further comprise projecting the acquired native posterior tibial slope axis on a sagittal plane relative to the patient for use as a reference axis; and visually displaying an acquired native posterior tibial slope axis in an intra-operative implant planning view.

A second aspect of the disclosure provides a method for planning a total knee arthroplasty (TKA) procedure on a patient, comprising obtaining a pre-operative image of a knee of the patient; identifying, on the pre-operative image, an angle corresponding to a native posterior tibial slope angle of the patient; and planning a tibial cut plane based on the angle.

In certain embodiments, the tibial cut plane is planned such that, when a tibia of the patient is cut along the tibial cut plane, and a tibial tray is inserted onto a prepared proximal surface of the tibia, the tibial tray provides a posterior tibial slope corresponding to the native posterior tibial slope of the patient.

In certain embodiments, the pre-operative image is a lateral x-ray image.

In certain embodiments, identifying the angle comprises identifying a mechanical axis of a tibia of the patient; creating a first reference line perpendicular to the mechanical axis; drawing a second reference line aligned with the native posterior tibial slope of the patient; and measuring the angle between the first reference line and the second reference line, wherein the angle corresponds to the native posterior tibial slope angle.

Certain embodiments further comprise determining the second reference line relative to a lateral side of the tibia.

In certain embodiments, the steps of identifying the mechanical axis and creating the first reference line are performed automatically using a surgical computer platform.

In certain embodiments, the step of drawing the second reference line is performed manually by a user.

Certain embodiments further comprise entering the angle corresponding to the native tibial slope angle into a surgical computer platform as a patient-specific parameter for planning the TKA procedure.

Certain embodiments further comprise intra-operatively confirming the native posterior tibial slope by providing a camera tracking system adapted to track a pose of one or more tracking arrays, and a computer platform in communication with the camera tracking system; affixing a first tracking array to a tibia of the patient, wherein the first tracking array comprises a plurality of reference elements adapted for tracking by the camera tracking system; registering a pose of the tibia to the computer platform; providing a navigated instrument comprising a second tracking array having a plurality of reference elements thereon and adapted for tracking by the camera tracking system; using the navigated instrument, directly acquiring an axis corresponding to the native posterior tibial slope of the tibia; and confirming the position of the reference line as identified pre-operatively.

In certain embodiments, directly acquiring the axis comprises aligning a shaft of the navigated instrument with the native posterior tibial slope; and capturing an axis of the native posterior tibial slope in response to a user input.

A third aspect of the disclosure provides a method for planning a total knee arthroplasty (TKA) procedure on a patient, comprising providing a camera tracking system adapted to track a pose of one or more tracking arrays, a computer platform in communication with the camera tracking system, a first tracking array comprising a plurality of reference elements adapted for tracking by the camera tracking system, and a navigated instrument comprising a second tracking array having a plurality of reference elements thereon and adapted for tracking by the camera tracking system; affixing the first tracking array to a tibia of the patient; using the camera tracking system and the first tracking array, registering a pose of the tibia to the computer platform; creating a reference line on a visual representation of the tibia, wherein the reference line corresponds to a native posterior tibial slope of the patient; and intra-operatively planning a tibial cut plane based on a position of the reference line.

In certain embodiments, the navigated instrument comprises a navigated stylus.

In certain embodiments, creating the reference line comprises acquiring, on a side of evaluation of the tibia, a first point on a most prominent posterior aspect of a tibial plateau, and a second point on a most prominent anterior aspect of the tibial plateau.

In certain embodiments, the side of evaluation of the tibia comprises a medial side or a lateral side.

In certain embodiments, creating the reference line further comprises establishing the reference line between the first point and the second point on the visual representation of the tibia.

In certain embodiments, creating the reference line further comprises projecting the reference line onto a sagittal plane relative to the patient.

Certain embodiments further comprise displaying the tibia to the user in real time on a display device to provide the visual representation of the tibia, wherein the display device is in communication with the computer platform and the camera tracking system.

Certain embodiments further comprise displaying the native posterior tibial slope of the patient in an intra-operative implant planning view on the display device.

In certain embodiments, the tibial cut plane is planned such that, when the tibia of the patient is cut along the tibial cut plane, and a tibial tray is inserted onto a prepared proximal surface of the tibia, the tibial tray provides a tibial slope corresponding to the native posterior tibial slope of the patient.

A fourth aspect of the disclosure provides a method for planning a total knee arthroplasty (TKA) procedure on a patient, comprising providing a camera tracking system adapted to track a pose of one or more tracking arrays, a computer platform in communication with the camera tracking system, a first tracking array comprising a plurality of reference elements adapted for tracking by the camera tracking system, and a navigated instrument comprising a second tracking array having a plurality of reference elements thereon and adapted for tracking by the camera tracking system; affixing the first tracking array to a tibia of the patient; registering a pose of the tibia to the computer platform; using the navigated instrument, directly acquiring an axis corresponding to a native posterior tibial slope of the tibia; and intra-operatively planning a tibial cut plane based on a position of the axis.

In certain embodiments, the navigated instrument comprises a navigated stylus.

In certain embodiments, the navigated instrument comprises a navigated tibial wall hook instrument.

In certain embodiments, directly acquiring the axis comprises aligning a shaft of the navigated instrument with the native posterior tibial slope, such that the shaft is tangent to a posterior tibial plateau.

Certain embodiments further comprise performing the aligning on a medial side or a lateral side of the tibia.

In certain embodiments, directly acquiring the axis further comprises capturing an axis of the native posterior tibial slope in response to a user input when the shaft of the navigated instrument is tangent to the posterior tibial plateau.

In certain embodiments, the user input is provided via a foot pedal in communication with the computer platform.

In certain embodiments, the user input is provided via a button provided in a user interface displayed on a display device in communication with the computer platform.

Certain embodiments further comprise projecting the acquired axis on a sagittal plane relative to the patient; and visually displaying the axis on a display device in an intra-operative implant planning view.

In certain embodiments, the tibial cut plane is planned such that, when the tibia is cut along the tibial cut plane, and a tibial tray is inserted onto a prepared proximal surface of the tibia, the tibial tray provides a posterior tibial slope corresponding to the native posterior tibial slope of the patient.

In certain embodiments, intra-operatively planning the tibial cut plane further comprises selecting a tibial insert component for insertion based on the position of the axis.

These and other aspects, advantages and salient features of the disclosure will become apparent from the following detailed description, which, when taken in conjunction with the annexed drawings, describe embodiments of the disclosure.

### BRIEF DESCRIPTION OF THE DRAWINGS

Aspects of the present disclosure are illustrated by way of example and are not limited by the accompanying drawings.

FIGS. 1A, 1B, and 1C illustrate constitutional, systematic, and kinematic alignment techniques for TKA procedures, respectively.

FIG. 2 illustrates varus and valgus rotation of a femoral implant in a TKA procedure according to certain embodiments.

FIG. 3 illustrates varus and valgus rotation of a tibial implant in a TKA procedure according to certain embodiments.

FIG. 4 illustrates internal and external rotation of a femoral implant in a TKA procedure according to certain embodiments.

FIG. 5 illustrates the parameter of tibial posterior slope in a TKA procedure according to certain embodiments.

FIG. 6 provides a coronal slice view of portions of a distal femur and femoral implant in a TKA procedure according to certain embodiments.

FIG. 7 provides a coronal slice view of portions of a proximal tibia and tibial tray in a TKA procedure according to certain embodiments.

FIG. 8 provides a transverse slice view of portions of a distal femur and femoral implant in a TKA procedure according to certain embodiments.

FIG. 9 illustrates a surgical system according to certain embodiments.

FIG. 10 illustrates a surgical robot component of the surgical system of FIG. 9 according to certain embodiments.

FIG. 11 illustrates a camera tracking system component of the surgical system of FIG. 9 according to certain embodiments.

FIG. 12 illustrates a passive end effector that is connectable to an arm of the surgical robot of FIG. 10 according to certain embodiments.

FIG. 13 illustrates a medical operation in which a surgical robot and a camera system, as shown in FIGS. 10-11, are disposed around a patient in accordance with certain embodiments.

FIG. 14 illustrates a block diagram of components of a surgical system according to certain embodiments.

FIG. 15 illustrates a block diagram of a surgical system computer platform that includes a surgical planning computer, which may be separate from and operationally connected to a surgical robot, or at least partially incorporated therein, according to certain embodiments.

FIGS. 16-17 illustrate processes in a 3D-image based workflow for planning a TKA procedure according to certain embodiments.

FIGS. 18A and 18B illustrate sagittal slice views of a proximal tibia, including the patient's native tibial slope (FIG. 18A) and the tibial slope following tibial plateau resection and tibial tray insertion (FIG. 18B) according to the workflows depicted in FIGS. 16-17.

FIGS. 19-20 illustrate processes in a 2D-image based workflow for planning a TKA procedure according to certain embodiments.

FIG. 21 illustrates a lateral x-ray image of the proximal tibia and aspects of a process for determining the patient's native posterior tibial slope according to the workflows depicted in FIGS. 19-20.

FIG. 22 illustrates processes in an imageless workflow for determining a patient's native posterior tibial slope via intra-operative point acquisition in accordance with certain embodiments.

FIG. 23 illustrates aspects of the imageless workflow depicted in FIG. 22, in accordance with certain embodiments.

FIG. 24 illustrates processes in an imageless workflow for determining a patient's native posterior tibial slope via intra-operative direct axis acquisition in accordance with certain embodiments.

FIG. 25 illustrates aspects of the imageless workflow depicted in FIG. 24, in accordance with certain embodiments.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE DISCLOSURE

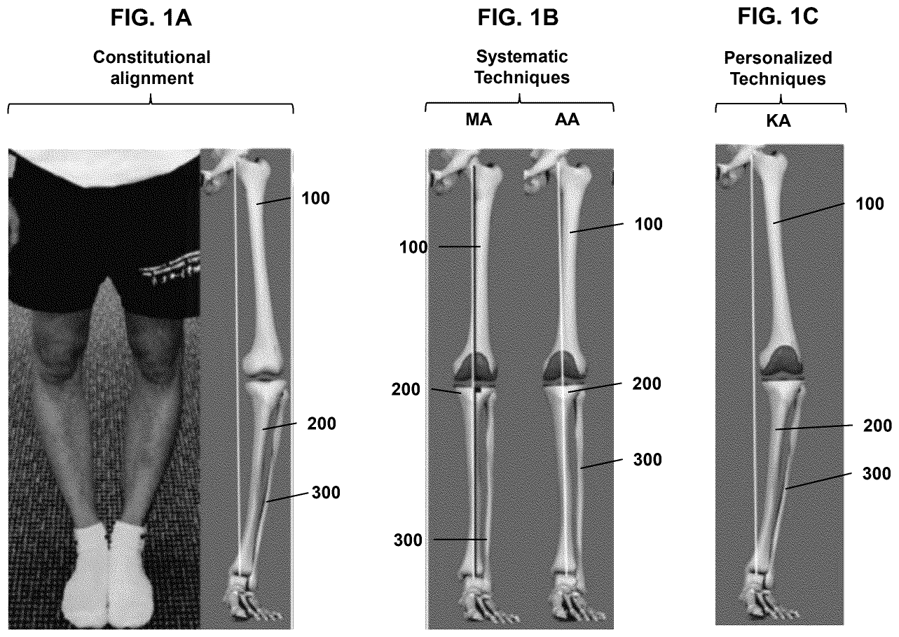

The disclosure provides methods, systems, and devices for planning tibial and femoral implant placement in TKA procedures to yield kinematic alignment (KA) of the post-operative patient knee. Kinematic alignment is depicted in FIG. 1C, and is distinguishable from other alignment techniques such as, e.g., mechanical alignment in goals and outcomes.

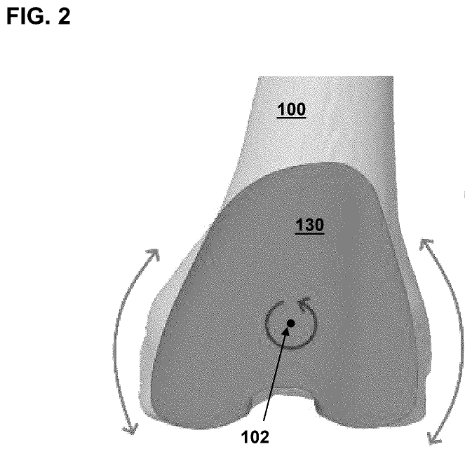

FIGS. 2-5 illustrate a set of parameters to be adjusted during the femoral and tibial component planning phase in order to perform a kinematic alignment (KA)-TKA.

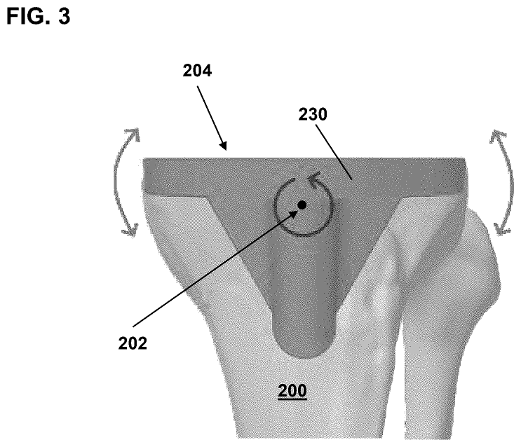

Referring to FIG. 2, one such parameter includes the varus/valgus rotation of the femoral component 130 relative to the femur 100. With the patient's affected leg in extension, the varus/valgus rotation of the femoral component (or, implant) 130 is characterized by a rotation of the implant 130 around the anterior-posterior axis 102 orthogonal to the patient coronal plane (not shown). Similarly, FIG. 3 illustrates the varus/valgus rotation of the tibial component 230 relative to the tibia 200. With the patient's affected leg in extension, the varus/valgus rotation of the tibial component 230 is characterized by a rotation of the implant 230 around the anterior-posterior axis 202 orthogonal to the patient coronal plane.

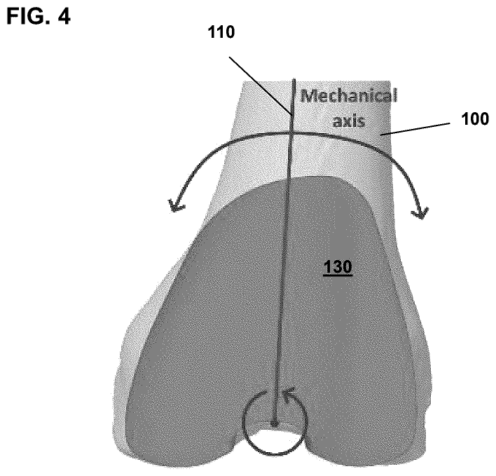

Referring to FIG. 4, another parameter includes the internal/external rotation of the femoral implant 130, which is characterized by a rotation of the femoral implant 130 around the mechanical axis 110 of the femur 100. When the rotation is performed in a direction toward the lateral patient side, the rotation is referred to as external, while rotation performed in the direction of the medial patient side is referred to as internal.

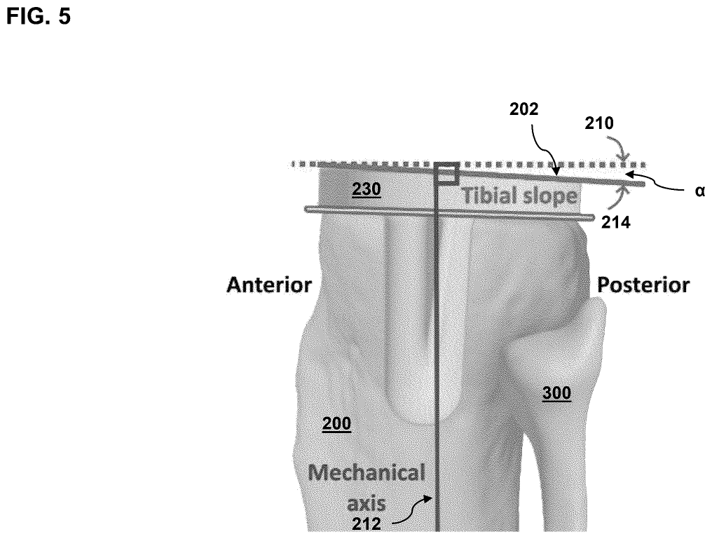

A further parameter, tibial posterior slope, is illustrated in FIG. 5. The tibial posterior slope is defined as the angle α between the horizontal antero-posterior axis 210, and the line 214 representing the posterior inclination of the tibial plateau 204 (FIG. 3). The horizontal antero-posterior axis 210 is defined as being perpendicular to the tibial mechanical axis 212. The posterior tibial slope 214 has an impact on flexion gap, knee joint stability, and posterior femoral rollback, which are related to a wide range of knee motions. Accordingly, planning and surgically achieving a kinematic alignment requires a careful measurement of the native tibial slope 214 on the patient anatomy.

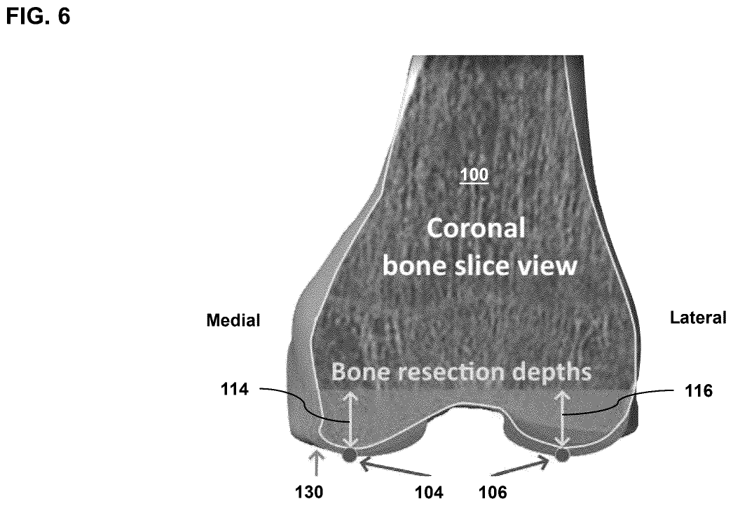

FIGS. 6-8 depict methods for adjusting the patient-specific biomechanical parameters described above and shown in FIGS. 2-4, namely, varus/valgus rotation of the femoral implant 130 and tibial implant 230, and internal/external rotation of the femoral implant 130. These adjustments contribute to the achievement of kinematic alignment (KA) of knee components in the TKA.

Referring to FIG. 6, adjustment of the varus/valgus rotation of the femoral implant 130 may be performed by evaluating the femoral distal condylar points 104, 106. The goal of the adjustment is to equalize implant resection depths 114, 116 with respect to medial and lateral distal condylar points 104, 106, respectively. This adjustment can be determined during the planning phase on pre-operative images such as, e.g., coronal slice views of a 3D bone model, or intra-operatively without images using points acquired at the surface of the bone.

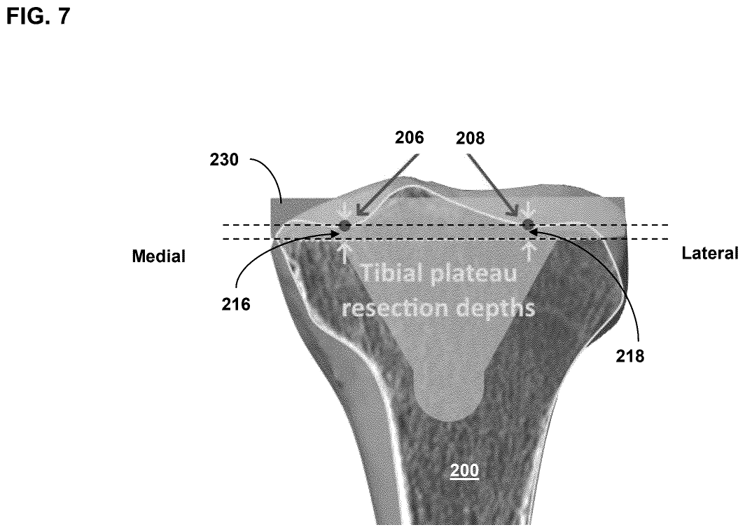

Referring to FIG. 7, adjustment of the varus/valgus ration of the tibial implant 230 may be performed by evaluating the deepest proximal plateau points 206, 208. The goal of the adjustment is to equalize the tibial plateau resection depths 216, 218 with respect to the medial and lateral deepest plateau points 206, 208. This adjustment can be determined during the planning phase on pre-operative images such as, e.g., coronal slice views of a 3D bone model, or intra-operatively without images using points acquired at the surface of the bone.

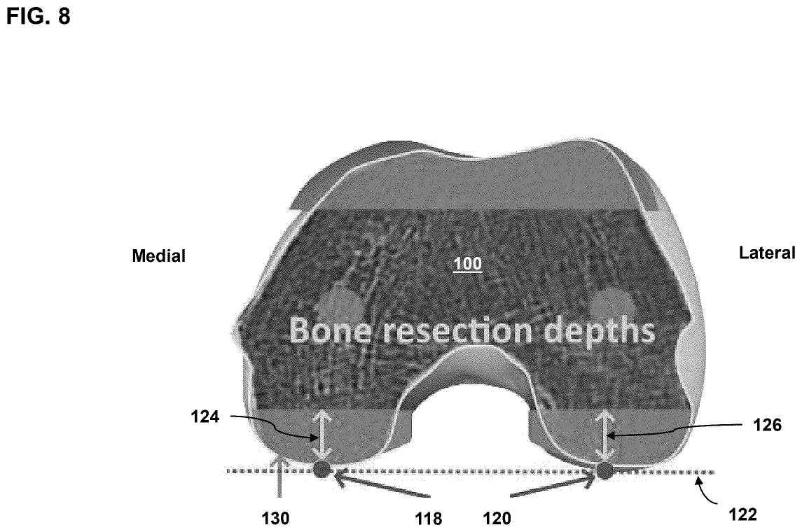

Turning to FIG. 8, adjustment of the internal/external rotation may be performed by evaluating the femoral posterior condylar points 118, 120. The goal of the adjustment is to set the femoral implant 130 to a neutral internal/external rotation with respect to the Posterior Condylar Axis (PCA) 122. Posterior resection depths 124, 126 are uniformized with respect to the medial and lateral posterior condylar points 118, 120.

Various embodiments described herein provide workflows, systems, and devices adapted to measure a patient's native posterior tibial slope (FIG. 5), which may then be used to plan KA-TKA procedures. Such planning may incorporate the foregoing parameter adjustments together with the patient's native posterior tibial slope, and may include planning resection planes and implant placement based on the measured posterior tibial slope.

### Surgical System

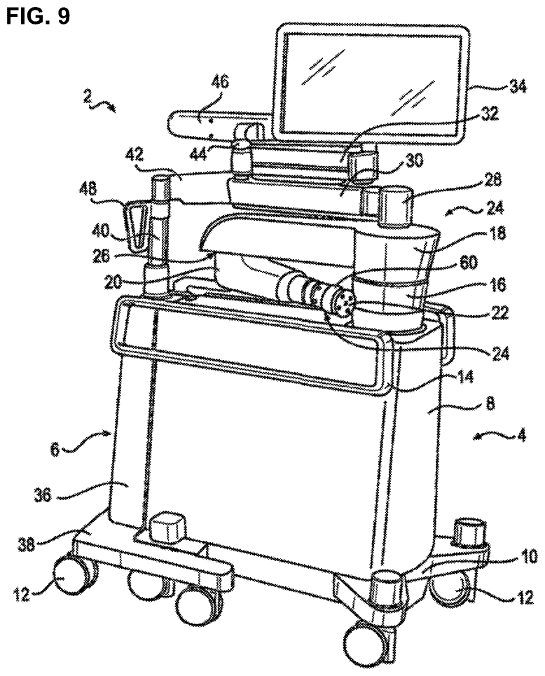

With reference to FIGS. 9-15, a surgical system including a computer platform and surgical robot may be used to plan and execute the adjustments to the parameters described herein. An exemplary surgical system may include a surgical system as described in U.S. Pat. No. 11,864,857, entitled “Surgical Robot with Passive End Effector (issued Jan. 9, 2024 to Globus Medical, Inc., Audubon, PA, US), which is incorporated by reference herein as though fully set forth.

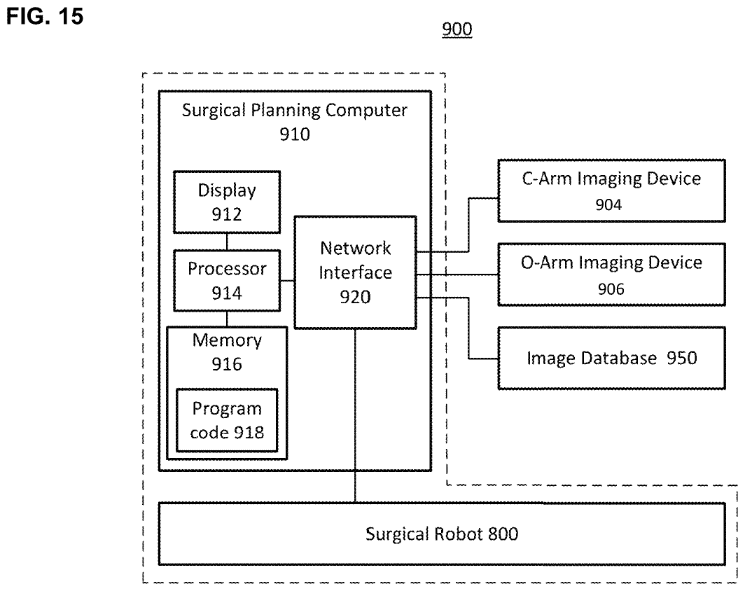

With reference to the figures, FIG. 9 illustrates an embodiment of an exemplary surgical system 2. Prior to performance of an orthopedic surgical procedure, a three-dimensional (“3D”) image scan may be taken of a planned surgical area of a patient using, e.g., a C-Arm imaging device 104 (FIG. 15), an O-Arm imaging device 106 (FIG. 15), or another medical imaging device such as a computed tomography (CT) image or MRI. This image can be obtained pre-operatively, e.g., weeks before a surgical procedure, or intra-operatively. Any known 3D or 2D image scan may be used in accordance with various embodiments of the surgical system 2. The image scan is sent to a computer platform in communication with the surgical system 2, such as the surgical system computer platform 900 of FIG. 15 which includes the surgical robot 800 (e.g., robot 2 in FIG. 9) and a surgical planning computer 910. A surgeon reviewing the image scan(s) on a display device 912 of the surgical planning computer 910 (FIG. 15) generates a surgical plan defining various planned aspects of the procedure including, e.g., a target or planned tibial cut plane. This plane is a function of patient anatomy constraints, selected implant and its size. In some embodiments, the surgical plan defining the target plane is planned on the image scan displayed on a display device.

The surgical system 2 of FIG. 9 may include a surgical robot 4 and a camera tracking system 6. Surgical robot 4 may be positioned adjacent the patient at any suitable location to properly assist medical personnel. Camera tracking system 6 may be positioned at the base of the patient, at patient shoulders, or any other location suitable to track the present pose and movement of the surgical robot 4 and the patient. Surgical robot 4 and camera tracking system 6 may be powered by an onboard power source and/or plugged into an external wall outlet.

Surgical robot 4 may be used to assist a surgeon by holding and/or using tools during a medical procedure. To properly utilize and hold tools, surgical robot 4 may rely on a plurality of motors, computers, and/or actuators to function. As illustrated in FIG. 9, robot body 8 may act as the structure in which the plurality of motors, computers, and/or actuators may be secured within surgical robot 4. Robot body 8 may also provide support for robot telescoping support arm 16. A robot base 10 may act as a lower support for surgical robot 4, and may in some embodiments attach robot body 8 to a plurality of wheels 12, at least one of which may be powered and motorized. A robot railing 14 may be provided to facilitate moving surgical system 2 without grasping robot body 8.

Robot body 8 may provide support for a Selective Compliance Articulated Robot Arm (“SCARA”) 24, which may be beneficial to use within the surgical system 2 due to the repeatability and compactness of the robotic arm. SCARA 24 may comprise robot telescoping support 16, robot support arm 18, and/or robot arm 20. Robot telescoping support 16 may be disposed along robot body 8, and may provide support for the SCARA 24 and display 34. In some embodiments, robot telescoping support 16 may extend and contract in a vertical direction.

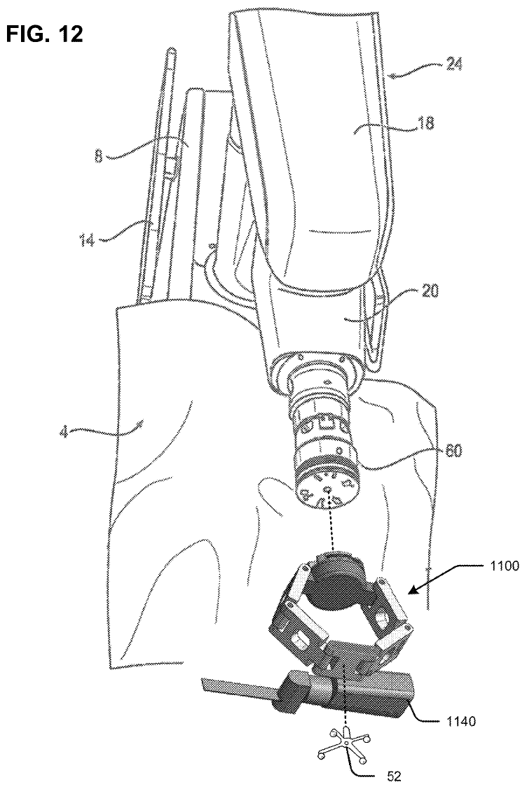

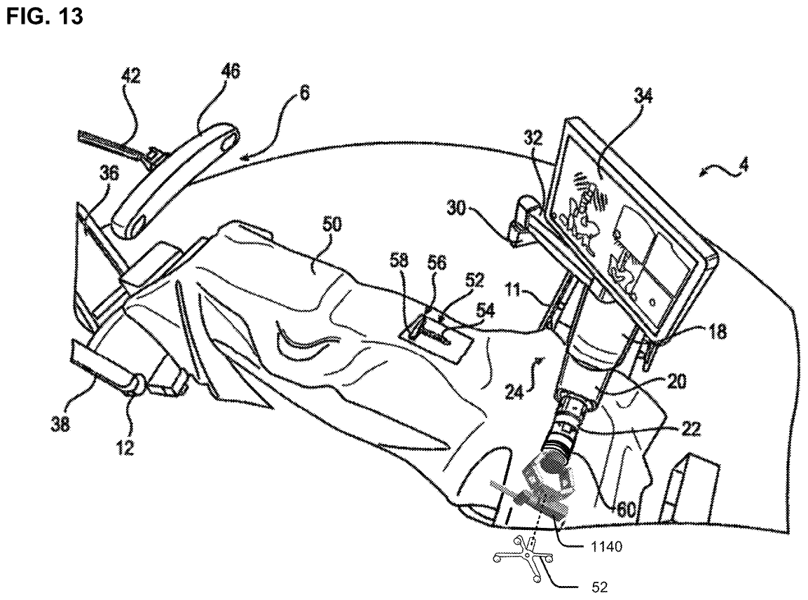

In some embodiments, medical personnel may move SCARA 24 through a command submitted by the medical personnel. The command may originate from input received on display 34, e.g., a touch screen, a tablet, or the actuation of one or more switches. An activation assembly 60 may include a switch and/or a plurality of switches. The activation assembly 60 may be operable to transmit a move command to the SCARA 24 allowing an operator to manually manipulate the SCARA 24. When the switch, or plurality of switches, is depressed the medical personnel may have the ability to move SCARA 24 easily. Additionally, when the SCARA 24 is not receiving a command to move, the SCARA 24 may lock in place to prevent accidental movement by personnel and/or other objects. By locking in place, the SCARA 24 provides a solid platform upon which a passive end effector 1100 and connected surgical instrument such as, e.g., surgical saw 1140 shown in FIGS. 12-13, are ready for use in a medical operation. The passive end effector 1100 in FIGS. 12-13 may attach to robot arm 20 in any suitable location, as described in U.S. Pat. No. 11,864,857, previously incorporated by reference, and may include an end effector coupler 22 to couple a base of the end effector 1100 to the robot arm 20. The passive end effector may in turn couple at a distal end thereof to a surgical instrument such as, e.g., a surgical saw 1140. The surgical saw 1140 may be configured to oscillate the saw blade for cutting in a constrained cutting plane of the saw blade, parallel to the working plane. In some embodiments, a dynamic reference array 52 is attached to the passive end effector 1100, e.g., to a tool attachment mechanism, and/or is attached to the surgical instrument. As described in U.S. Pat. No. 11,864,857, previously incorporated by reference, the system may enable force-controlled movement to allow the operator to move SCARA 24 and end effector coupler 22 easily and without large amounts of exertion.

Dynamic reference arrays, also referred to as “DRAs” herein, are rigid bodies which may be disposed on a patient, the surgical robot, the passive end effector, and/or the surgical instrument in a navigated surgical procedure. The camera tracking system 6 or other 3D localization system is configured to track in real-time the pose (e.g., positions and rotational orientations) of tracking markers of the DRA. The tracking markers may include the illustrated arrangement of balls or other optical markers. This tracking of 3D coordinates of tracking markers can allow the surgical system 2 to determine the pose of the DRA 52 in any space in relation to the target anatomical structure of the patient 50 in FIG. 13.

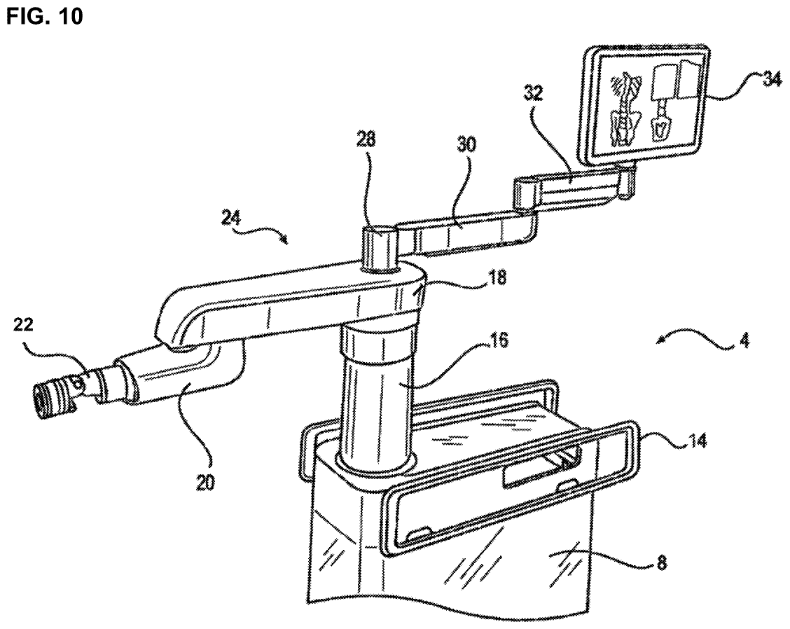

As illustrated in FIG. 9, a light indicator 28 may be positioned on top of the SCARA 24. Light indicator 28 may illuminate as any type of light to indicate “conditions” in which surgical system 2 is currently operating. For example, the illumination of green may indicate that all systems are normal. Illuminating red may indicate that surgical system 2 is not operating normally. A pulsating light may mean surgical system 2 is performing a function. Light and pulsation patterns may be combined without limitation to communicate current operating conditions, states, or other operational indications. In some embodiments, the light may be produced by LED bulbs, which may form a ring around light indicator 28. Light indicator 28 may be attached to lower display support 30. Lower display support 30, as illustrated in FIG. 10, may allow an operator to maneuver display 34 to any suitable location. Upper display support 32 may attach to lower display support 30, and may allow display 34 to rotate three hundred and sixty degrees in relation to upper display support 32. Likewise, upper display support 32 may rotate three hundred and sixty degrees in relation to lower display support 30. Display 34 may be any device which may be supported by upper display support 32. In embodiments, as illustrated in FIGS. 10 and 13, display 34 may produce color and/or black and white images.

In embodiments, a tablet may be used in conjunction with display 34 and/or without display 34. In embodiments, the tablet may be disposed on upper display support 32, in place of display 34, and may be removable from upper display support 32 during a medical operation. In addition, the tablet may communicate with display 34. The tablet may be able to connect to surgical robot 4 by any suitable wireless and/or wired connection. In some embodiments, the tablet may be able to program and/or control surgical system 2 during a medical operation. When controlling surgical system 2 with the tablet, all input and output commands may be duplicated on display 34. The use of a tablet may allow an operator to manipulate surgical robot 4 without having to move around patient 50 and/or to surgical robot 4.

As illustrated in FIG. 13, camera tracking system 6 works in conjunction with surgical robot 4 through wired or wireless communication networks. Referring to FIGS. 9 and 13, camera tracking system 6 can include some similar components to the surgical robot 4. For example, camera body 36 may provide the functionality found in robot body 8. Robot body 8 may provide the structure upon which camera 46 is mounted. The structure within robot body 8 may also provide support for the electronics, communication devices, and power supplies used to operate camera tracking system 6. Camera tracking system 6 may communicate directly to the tablet and/or display 34 by a wireless and/or wired network to enable the tablet and/or display 34 to control the functions of camera tracking system 6.

Camera body 36 is supported by camera base 38. Camera base 38 may function as robot base 10. In the embodiment of FIG. 9, camera base 38 may be wider than robot base 10, which may allow for camera tracking system 6 to connect with surgical robot 4. As illustrated in FIG. 9, the width of camera base 38 may be large enough to fit outside robot base 10. When camera tracking system 6 and surgical robot 4 are connected, the additional width of camera base 38 may allow surgical system 2 additional maneuverability and support for surgical system 2.

As with robot base 10, a plurality of powered wheels 12 may attach to camera base 38. Powered wheels 12 may allow camera tracking system 6 to stabilize and level or set fixed orientation in regards to patient 50, similar to the operation of robot base 10 and powered wheels 12. This stabilization may prevent camera tracking system 6 from moving during a medical procedure and may keep camera 46 from losing track of one or more DRAs 52 connected to an anatomical structure 54 and/or tool 58 within a designated area 56 as shown in FIG. 13.

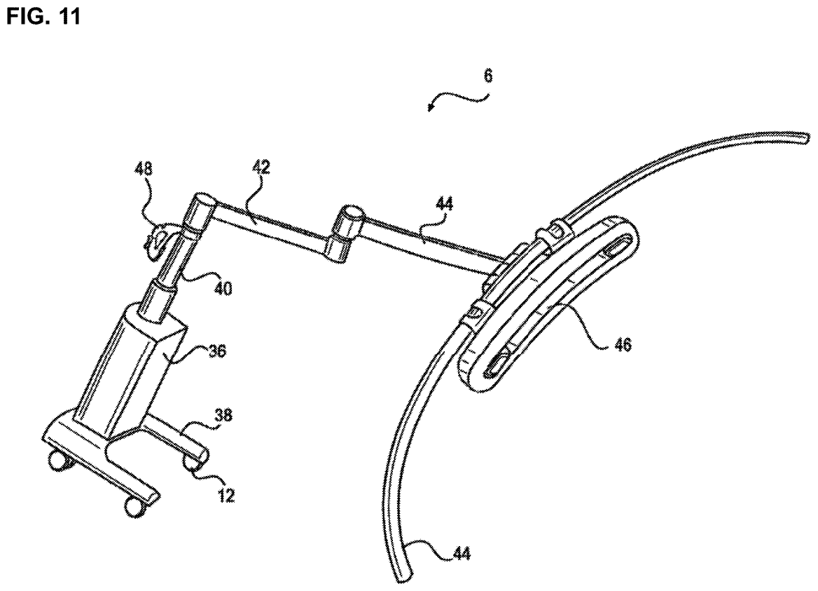

Camera telescoping support 40 may support camera 46. In embodiments, telescoping support 40 may move camera 46 higher or lower in the vertical direction, e.g., Camera handle 48 may be attached to camera telescoping support 40 at any suitable location. Lower camera support arm 42 may attach to camera telescoping support 40 at any suitable location, and may freely rotate three hundred and sixty degrees around telescoping support 40, allowing an operator to position camera 46 in any suitable location. Camera 46 may pivot in any direction at the attachment area between camera 46 and lower camera support arm 42. As shown in FIG. 11, a curved rail 44 may be disposed on lower camera support arm 42. Camera 46 may be moveably disposed along curved rail 44, and attached to curved rail 44 by any suitable mechanism such as, e.g., rollers, brackets, braces, motors, and/or any combination thereof, to allow camera 46 to move along curved rail 44.

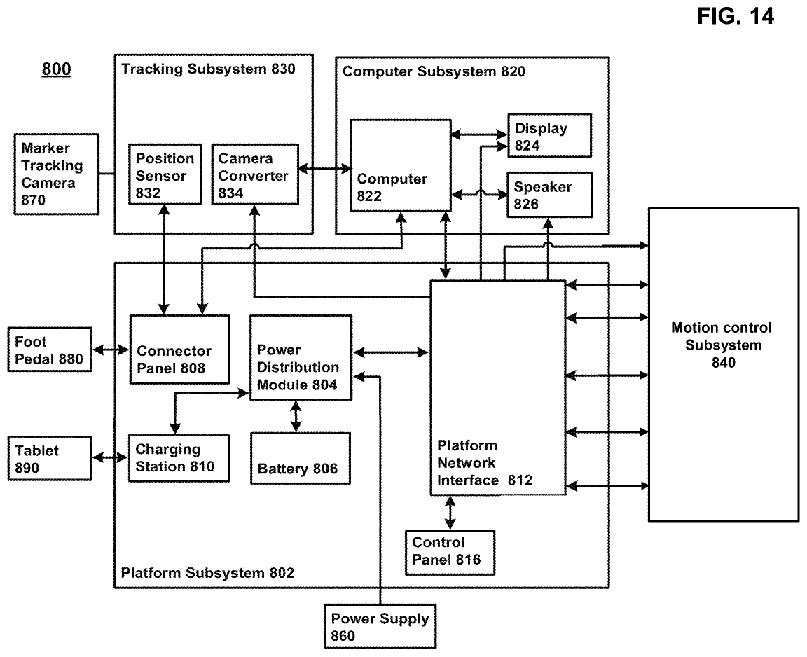

FIG. 14 illustrates a block diagram of components of a surgical system 800 configured according to some embodiments of the present disclosure, and which may correspond to the surgical system 2 above. Surgical system 800 includes platform subsystem 802, computer subsystem 820, motion control subsystem 840, and tracking subsystem 830. Platform subsystem 802 includes battery 806, power distribution module 804, connector panel 808, and charging station 810. Computer subsystem 820 includes computer 822, display 824, and speaker 826. Motion control subsystem 840 interacts with one or more load sensors in the end effector coupler 22, as described in U.S. Pat. No. 11,864,857, previously incorporated by reference. Tracking subsystem 830 includes position sensor 832 and camera converter 834. Surgical system 800 may also include a removable foot pedal 880 and removable tablet computer 890.

Input power is supplied to surgical system 800 via a power source 860 which may be provided to power distribution module 804. Power distribution module 804 receives input power and is configured to generate different power supply voltages that are provided to other modules, components, and subsystems of surgical system 800. Power distribution module 804 may be configured to provide different voltage supplies to connector panel 808, which may be provided to other components such as, e.g., computer 822, display 824, speaker 826, camera converter 834 and other components for surgical system 800. Power distribution module 804 may also be connected to battery 806, which serves as temporary power source in the event that power distribution module 804 does not receive power from an input power. At other times, power distribution module 804 may serve to charge battery 806.

Connector panel 808 may serve to connect different devices and components to surgical system 800 and/or associated components and modules. Connector panel 808 may contain one or more ports that receive lines or connections from different components. For example, connector panel 808 may have a ground terminal port that may ground surgical system 800 to other equipment, a port to connect foot pedal 880, a port to connect to tracking subsystem 830, which may include position sensor 832, camera converter 834, and marker tracking cameras 870. Connector panel 808 may also include other ports to allow communications via USB, Ethernet, HDMI, etc. to other components, such as computer 822.

Control panel 816 may provide various buttons or indicators that control operation of surgical system 800 and/or provide information from surgical system 800 for observation by an operator. For example, control panel 816 may include buttons to power on or off surgical system 800, lift or lower robot telescoping support 16, and lift or lower stabilizers that may be designed to engage wheels 12 to lock surgical system 800 from physically moving, e.g., via motion control subsystem 840. Other buttons may stop surgical system 800 in the event of an emergency, which may remove all motor power and apply mechanical brakes to stop all motion from occurring. Control panel 816 may also have indicators notifying the operator of certain system conditions such as a line power indicator or status of charge for battery 806.

Computer 822 of computer subsystem 820 includes an operating system and software to operate assigned functions of surgical system 800. Computer 822 may receive and process information from other components (for example, tracking subsystem 830, platform subsystem 802, and/or motion control subsystem 840) in order to display information to the operator. Further, computer subsystem 820 may provide output through the speaker 826 for the operator. The speaker 826 may be part of the surgical robot 4, part of a head-mounted display component, or may reside within another component of the surgical system 2 (FIG. 9). The display 824 may correspond to the display 34 shown in FIGS. 9 and 10, or may be a head-mounted display which projects images onto a see-through display screen which forms an augmented reality (AR) image that is overlaid on real-world objects viewable through the see-through display screen.

Tracking subsystem 830 may include position sensor 832 and camera converter 834. Tracking subsystem 830 may correspond to the camera tracking system 6 of FIG. 11. The marker tracking cameras 870 operate with the position sensor 832 to determine the pose of DRAs 52 (FIG. 13). This tracking may be conducted in a manner consistent with the present disclosure including the use of infrared or visible light technology that tracks the location of active or passive elements of DRAs 52, such as LEDs or reflective markers, respectively. The location, orientation, and position, e.g., pose of structures having these types of markers, such as DRAs 52, is provided to computer 822 and which may be shown to an operator on display 824. For example, as shown in FIGS. 12-13, a surgical saw 1140 (or other instrument) having a DRA 52 or which is connected to an end effector coupler 22 having a DRA 52 tracked in this manner (which may be referred to as a navigational space) may be shown to an operator in relation to a 3D image of a patient's anatomical structure.

Motion control subsystem 840 may be configured to physically move vertical column 16, upper arm 18, lower arm 20, or rotate end effector coupler 22. The physical movement may be conducted through the use of one or more motors. The surgical planning computer 910 shown in FIG. 15 can provide control input to the motion control subsystem 840 to guide movement of the end effector coupler 22 to position a passive end effector, which is connected thereto, to a planned pose (i.e., location and angular orientation relative to defined 3D orthogonal reference axes) relative to an anatomical structure that is to be cut during a surgical procedure.

FIG. 15 illustrates a block diagram of a surgical system computer platform 900 that includes a surgical planning computer 910 which may be separate from and operationally connected to a surgical robot 800 or at least partially incorporated therein. Alternatively, at least a portion of operations or processes disclosed herein for the surgical planning computer 910 may be performed by components of the surgical robot 800 such as by the computer subsystem 820. The surgical planning computer 910 includes a display 912, at least one processor circuit (or “processor”) 914, at least one memory circuit (or “memory”) 916 containing computer readable program code 918, and at least one network interface 920. The network interface 920 can be configured to connect to a C-Arm imaging device 904, an O-Arm imaging device 906, another medical imaging device, an image database 950 of medical images, components of the surgical robot 800, and/or other electronic equipment.

When the surgical planning computer 910 is at least partially integrated within the surgical robot 800, the display 912 may correspond to, e.g., the display 34 of FIG. 10 and/or the tablet 890 of FIG. 14 and/or a head-mounted display, the network interface 920 may correspond to the platform network interface 812 of FIG. 14, and the processor 914 may correspond to the computer 822 of FIG. 14.

The processor 914 may include one or more data processing circuits, such as a general purpose and/or special purpose processor, e.g., microprocessor and/or digital signal processor. The processor 914 is configured to execute the computer readable program code 918 in the memory 916 to perform operations or processes, which may include some or all of the operations or processes described herein as being performed by a surgical planning computer.

The processor 914 can operate to display on the display device 912 an image of a bone that is received from, e.g., one of the imaging devices 904 and 906 and/or from the image database 950 through the network interface 920. The processor 914 receives an operator's definition of where an anatomical structure, i.e. one or more bones, shown in one or more images is to be cut, such as by an operator touch selecting locations on the display 912 for planned surgical cuts or using a mouse-based cursor to define locations for planned surgical cuts.

The surgical planning computer 910 enables anatomy measurement, useful for knee surgery, like measurement of various angles determining center of hip rotation, center of angles, natural landmarks (e.g. transepicondylar line, Whitesides line, and posterior condylar line), etc. Some measurements can be automatic while some others involve human input or assistance. This surgical planning computer 910 allows an operator to choose the correct implant for a patient, including choice of size and alignment. The surgical planning computer 910 enables automatic or semi-automatic (involving human input) segmentation (image processing) for CT images or other medical images. The surgical plan for a patient may be stored in a cloud-based server for retrieval by the surgical robot 800. During the surgery, the surgeon will choose which cut to make (e.g. posterior femur, proximal tibia etc.) using a computer screen (e.g. touchscreen) or augmented reality interaction via, e.g., a head-mounted display. The surgical robot 4 may automatically move the surgical saw blade to a planned position so that a target plane of planned cut is optimally placed within a workspace of the passive end effector, e.g., passive end effector 1100 (FIG. 12), interconnecting the surgical saw blade and the robot arm 20. Commands enabling movement can be given by user using various modalities, e.g. foot pedal 880 (FIG. 14).

In some embodiments, the surgical system computer platform 900 can use two DRAs to track patient anatomy position, e.g., one on each of the tibia and the femur of a patient. The platform 900 may use standard navigated instruments for the registration and checks (e.g., a pointer similar to the one used in Globus ExcelsiusGPS system for spine surgery). Tracking markers allowing for detection of DRAs' movement in reference to tracked anatomy can be used as well.

The processor 914 may graphically illustrate on the display 912 one or more cutting planes intersecting the displayed anatomical structure at the locations selected by the operator for cutting the anatomical structure. The processor 914 also determines one or more sets of angular orientations and locations where the end effector coupler 22 should be positioned so a cutting plane of the surgical saw blade will be aligned with a target plane to perform the operator defined cuts, and stores the sets of angular orientations and locations as data in a surgical plan data structure. The processor 914 uses the known range of movement of the tool attachment mechanism of the passive end effector to determine where the end effector coupler 22 (FIG. 10) attached to the robot arm 20 needs to be positioned.

The computer subsystem 820 of the surgical robot 800 receives data from the surgical plan data structure and receives information from the camera tracking system 6 indicating a present pose of an anatomical structure that is to be cut and indicating a present pose of the passive end effector and/or surgical saw tracked through DRAs. The computer subsystem 820 determines a pose of the target plane based on the surgical plan defining where the anatomical structure is to be cut and based on the pose of the anatomical structure. The computer subsystem 820 generates steering information based on comparison of the pose of the target plane and the pose of the surgical saw. The steering information indicates where the passive end effector needs to be moved so the cutting plane of the saw blade becomes aligned with the target plane and the saw blade becomes positioned a distance from the anatomical structure to be cut that is within the range of movement of the tool attachment mechanism of the passive end effector.

### Patient Registration Workflows

Various workflows are provided to register the patient in the tracking space of the navigation system described above. Patient registration can include matching the patient anatomy with a numeric representation of the corresponding bone, such as a 3D model of the bone. The bone representation may be constructed from, e.g., a set of CT images (CT workflow), a set of fluoroscopy images, or based on a generic bone model (imageless workflow). In some embodiments of the present disclosure, the system, e.g., computer platform 900, may perform one of a number of available workflows to register a patient to the surgical system including the surgical robot 800 prior to surgery. Exemplary patient registration work flows are disclosed in U.S. patent application Ser. No. 18/184,192, filed Mar. 15, 2023 and published as US 2024/0020840 A1, entitled “Registration of 3D and 2D images for surgical navigation and robotic guidance without using radiopaque fiducials in the images,” which is incorporated by reference as though fully set forth herein.

### Measurement of Tibial Slope

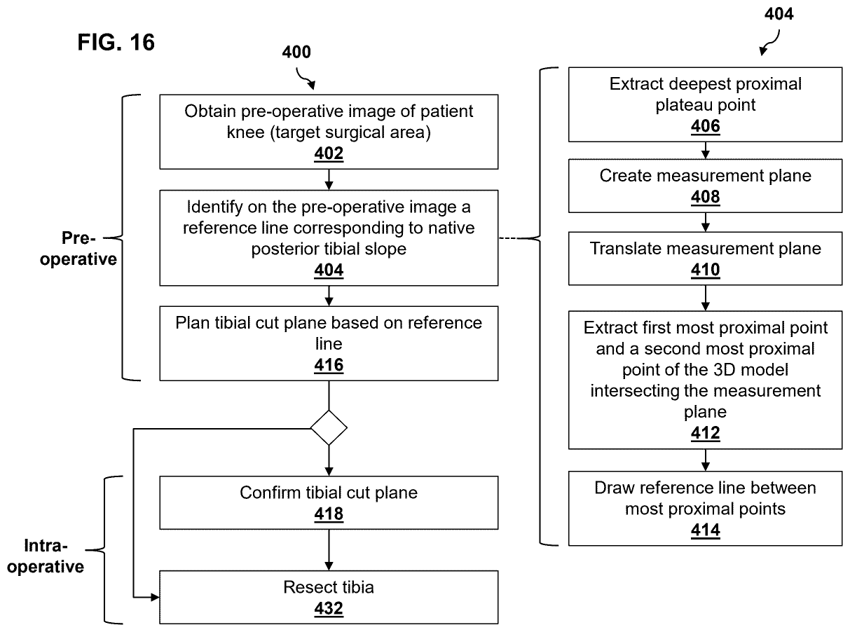

Referring to FIGS. 16-25, image-based workflows 400, 500 and imageless workflows 600, 700 are described herein for pre-operatively and/or intra-operatively determining a patient's native posterior tibial slope (PTS). These workflows may be used with a surgical system computer platform such as, e.g., computer platform 900 including surgical planning computer 910 and surgical robot 800, to plan and perform a KA-TKA procedure. Such planning may include, e.g., planning a tibial cut plane along which the tibia is to be resected during the procedure, and other aspects of the TKA procedure including, e.g., implant selection and placement. The TKA procedure may then be performed, e.g., by the surgeon with the aid of the surgical system 800, according to the pre-operatively or intra-operatively determined plan. The execution of the surgical plan may optionally include intra-operative steps to verify the pre-operatively determined native posterior tibial slope as discussed herein.

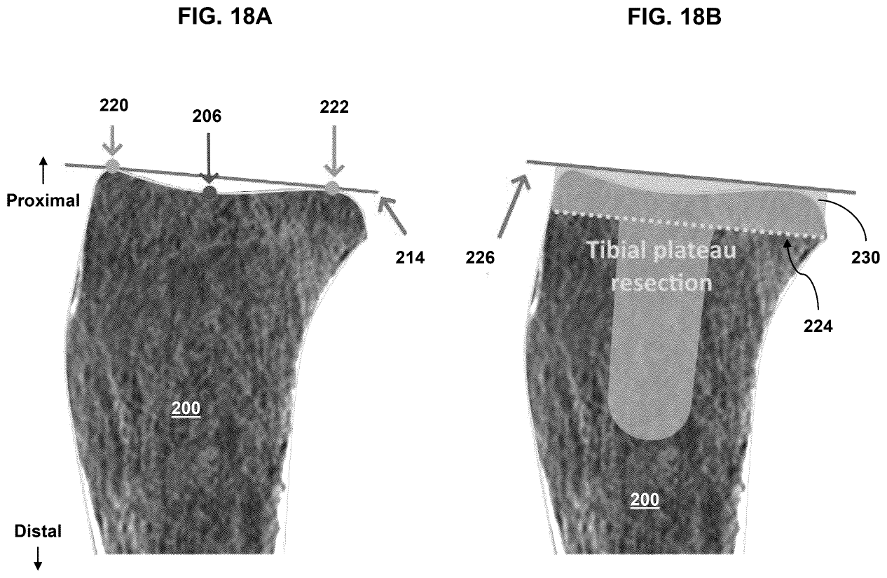

According to one embodiment, described with reference to FIGS. 16-18, a process is provided for planning and performing a KA-TKA procedure on a patient, using a 3D image-based workflow 400 for determining the patient's native posterior tibial slope (PTS) in the affected knee. Workflow 400 includes process 402 of obtaining a pre-operative image of a knee of the patient. The image may be captured any period of time prior to surgery, e.g., weeks, days, hours, or minutes prior to surgery, and may be a 3D image such as, e.g., a CT scan or an MRI. The image may be captured using e.g., an O-Arm imaging device 906 (FIG. 15) or another imaging device, and may be obtained for use with the surgical planning computer 910 directly from the imaging device (e.g., imaging device 906) or from a database 950 of medical images (FIG. 15). The pre-operative image may include the target surgical area, and may particularly include the proximal tibia 200, as shown in FIG. 18A. Regardless of source, the 3D image(s) (e.g., CT or MRI) are imported to the surgical planning computer 910, segmented, and a 3D bone model is generated. The 3D bone model may be displayed to the user, e.g., on display 912 (FIG. 15).

Referring back to the workflow of FIG. 16, process 404 includes identifying, on the pre-operative image (FIG. 18A), a reference line 214 corresponding to a native posterior tibial slope of the patient. Process 404 may include a number of subprocesses 406, 408, 410, 412, and 414.

Sub-process 406 includes extracting from the 3D bone model a deepest proximal plateau point. The deepest proximal plateau point 206 or 208 may be evaluated and extracted at either or both of the medial side (medial deepest proximal plateau point 206; FIG. 7, 18A) or the lateral side (lateral deepest proximal plateau point 208; FIG. 7) of the tibia 200, e.g., sub-process 406 may include extracting the medial deepest proximal plateau point 206 or the lateral deepest proximal plateau point 208. Certain embodiments may particularly include extracting the medial deepest proximal plateau point 206.

Subprocess 408 includes creating a measurement plane parallel to a sagittal plane of the patient.

Subprocess 410 includes translating the measurement plane to the deepest proximal plateau point 206 or 208 corresponding to the side of evaluation, e.g., the medial side or the lateral side respectively. The side of evaluation corresponds to the side of the tibia on which the deepest proximal plateau point (206 or 208) was extracted.

Subprocess 412 includes extracting a first most proximal point 220 and a second most proximal point 222 of the 3D bone model (FIG. 18A) that intersect the measurement plane created in subprocess 408 and translated in subprocess 410.

In certain embodiments, the extracting, e.g. in subprocesses 406, 412 is performed automatically by the surgical planning computer 910, while in others the extracting may be performed manually by the user, e.g., via visual identification of the respective points on the image displayed on display 912, and user input provided to surgical planning computer 910 via a user interface.

Subprocess 414 includes drawing the reference line 214 on the pre-operative image (FIG. 18A) between the first most proximal point 220 and the second most proximal point 222. The reference line 214 corresponds to the patient's native posterior tibial slope (PTS).

Process 416 includes planning a tibial cut plane 224 based on the reference line 214. In particular, the tibial cut plane 224 is planned such that, when the tibia 200 is cut along the tibial cut plane 224, and a tibial tray 230 is inserted onto a prepared proximal surface of the tibia 200 as shown in FIG. 18B, the resulting posterior slope 226 of the tibial components (e.g., tibial tray 230) provides a PTS corresponding to, e.g., substantially replicating that of the native PTS of the patient as represented by reference line 214.

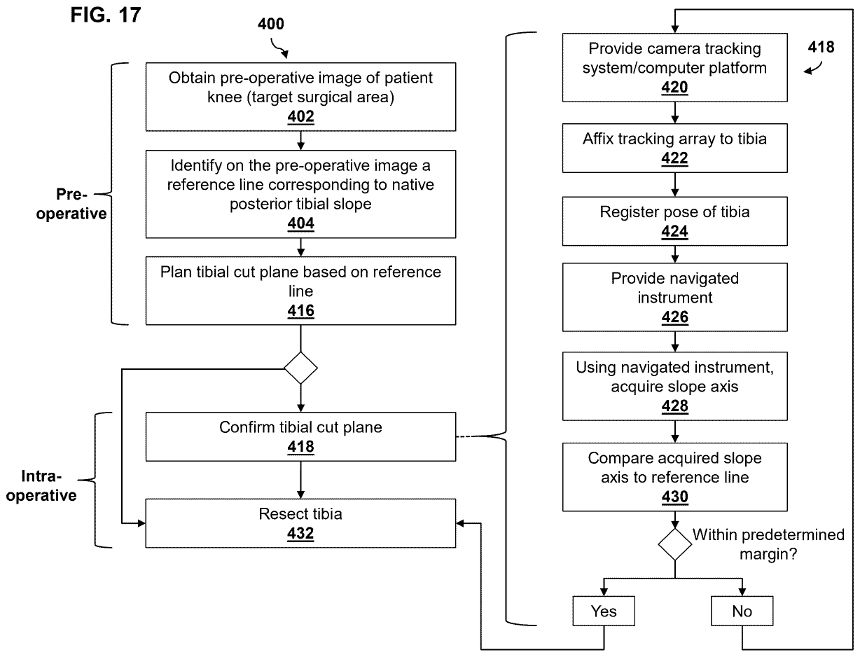

Referring to FIGS. 16-17, workflow 400 may additionally include an optional intra-operative process 418 to confirm the pre-operatively determined surgical plan, including the planned tibial cut plane. Process 418 may include sub-processes 420, 422, 424, 426, 428, and 430, as shown in FIG. 17.

Subprocess 420 provides a computer platform such as, e.g., computer platform 900 including a surgical planning computer 910 (FIG. 15), and a camera tracking system such as, e.g., camera tracking system 6 (FIG. 11) as described herein. The camera tracking system is adapted to track a pose of one or more tracking arrays such as, e.g., DRA 52 (FIGS. 12-13), and is in communication with the computer platform as described above.

Subprocess 422 includes making an incision in the patient's leg, and affixing a first tracking array such as, e.g., DRA 52 (FIGS. 12-13), to the relevant tibia of the patient. The first tracking array comprises a plurality of reference elements adapted for tracking by the camera tracking system, as described herein.

Subprocess 424 includes registering a pose of the tibia 200 to the computer platform, as described herein.

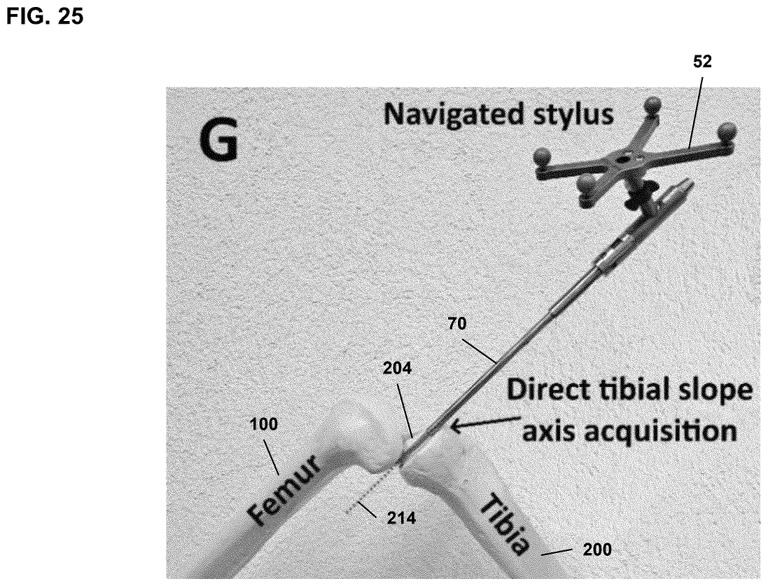

Subprocess 426 includes providing a navigated instrument 70 including a second tracking array such as a DRA 52 (FIG. 25), having a plurality of reference elements disposed thereon and adapted for tracking by the camera tracking system. The navigated instrument 70 may be, e.g., a navigated stylus, a pose of which may also be registered to the computer platform.

Subprocess 428, illustrated in FIG. 25, includes using the navigated instrument 70 to directly acquire an axis corresponding to a native PTS of the plateau 204 of the tibia 200, including aligning a shaft of the navigated instrument 70 with the native PTS (represented by line 214); and capturing an axis of the native PTS. The capturing may be performed by the computer platform in response to a user input, e.g., using a foot pedal or a user interface on a display.

Subprocess 430 includes comparing the slope axis acquired in subprocess 428 with the position of the reference line 214 as identified pre-operatively in process 404 (e.g., via subprocesses 406-414 in FIG. 16). The slope axis as acquired in subprocess 428 may be deemed to confirm or be consistent with the pre-operative plan if it is within a predetermined margin. The slope axis as acquired in subprocess 428 may be deemed to fail to confirm, to conflict with, or be inconsistent with the pre-operative plan if it is outside the predetermined margin. The predetermined margin may be, e.g., a margin of 1%, 2%, 5%, or similar. When the slope axis as acquired in subprocess 428 confirms the pre-operative determination of PTS and corresponding surgical plan, the TKA procedure may proceed as pre-operatively planned. When the slope axis as acquired in subprocess 428 fails to confirm the pre-operative determination of PTS and corresponding surgical plan, the surgical plan may be revised prior proceeding with the TKA procedure. Revision of the surgical plan may include, e.g., repeating processes 404 and/or 418 as needed. A further process may include projecting the acquired native posterior tibial slope axis on a sagittal plane relative to the patient for use as a reference axis; and visually displaying an acquired native posterior tibial slope axis in an intra-operative implant planning view.

Following planning of the tibial cut plane in process 416, and optionally following confirmation of the planned tibial cut plane in process 418, the TKA procedure may be performed, including process 432 of resecting the tibia in accordance with the surgical plan.

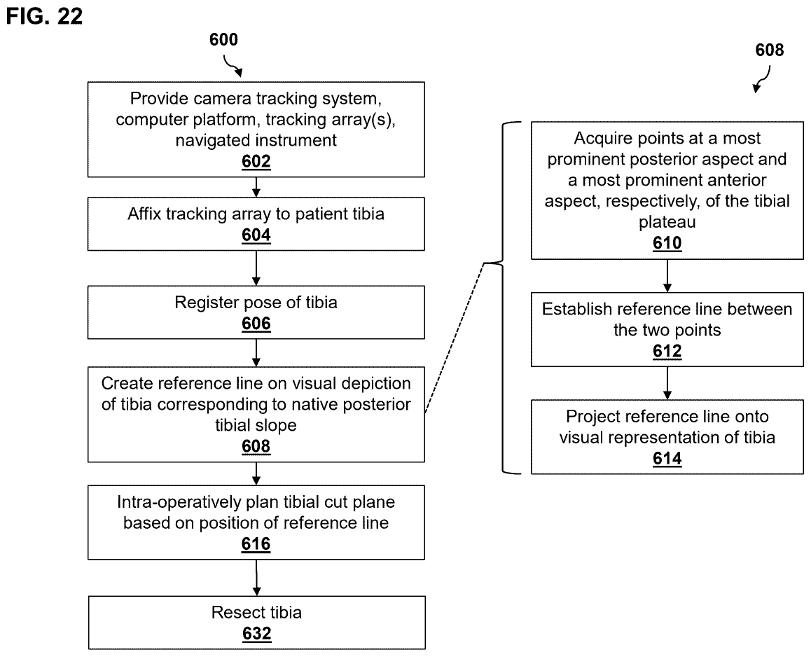

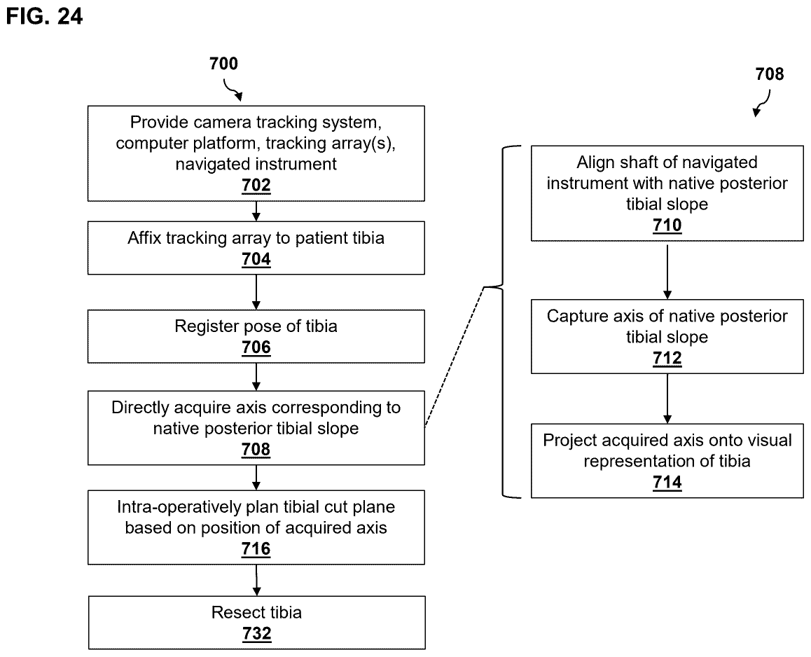

The foregoing optional confirmatory process 418 is substantially analogous to the process described herein below relative to workflow 700 (FIG. 24), in which direct acquisition of the axis corresponding to native PTS is used as a primary method of determining PTS. Alternatively or additionally, a process such as described herein below relative to workflow 600 (FIG. 22) may be deployed as a confirmatory process in workflow 400 following tibial cut plane planning process 416, in accordance with embodiments of the disclosure.

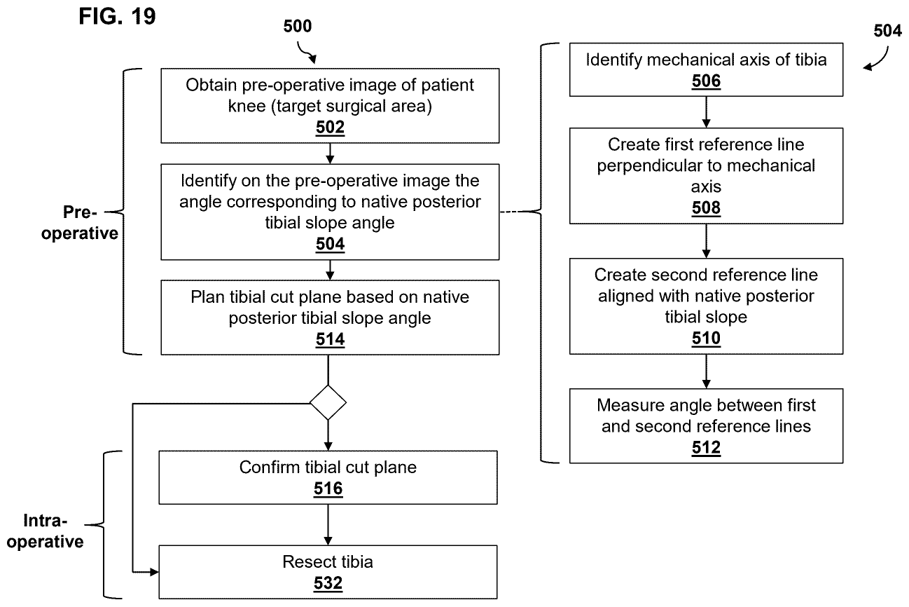

According to another embodiment, described with reference to FIGS. 19-21, a process is provided for performing a KA-TKA procedure on a patient, using an image-based workflow 500 for determining the patient's native posterior tibial slope in the affected knee.

According to another embodiment, described with reference to FIGS. 19-21, a process is provided for planning and performing a KA-TKA procedure on a patient, using a 2D image-based workflow 500 for determining the patient's native PTS.

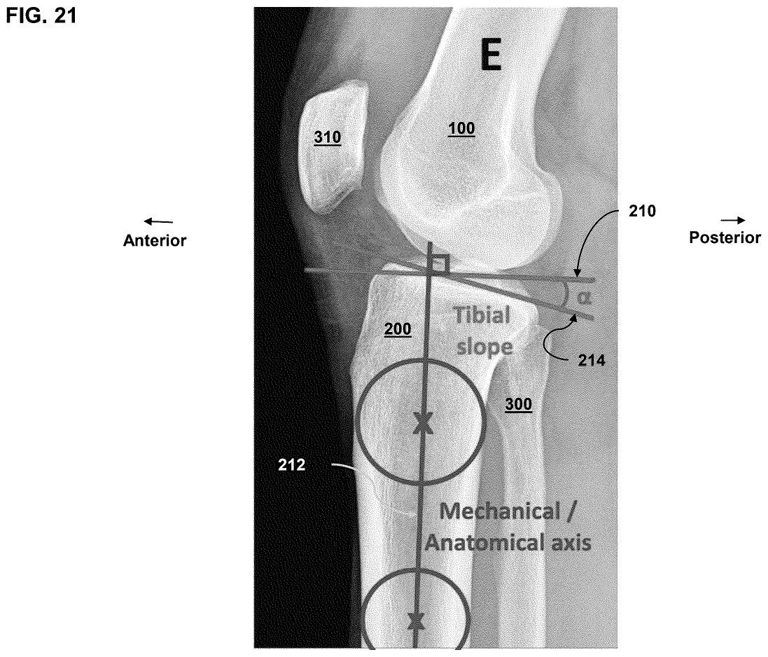

Workflow 500 includes process 502 of obtaining a pre-operative image of a knee of the patient. The image may be captured any period of time prior to surgery, e.g., weeks, days, hours, or minutes prior to surgery, and may be a 2D image such as, e.g., an x-ray. The image may be captured using e.g., a C-Arm imaging device 904 (FIG. 15) or another imaging device, and may be obtained for use with the surgical planning computer 910 directly from the imaging device (e.g., imaging device 904) or from a database 950 of medical images (FIG. 15). The pre-operative image may include the target surgical area, and may particularly include the proximal tibia 200, as shown in FIG. 21. Also shown are the distal femur 100, proximal fibula 300, and patella 310. In some embodiments, the pre-operative image is a lateral x-ray image as shown in FIG. 21. The 2D image(s) may be displayed to the user, e.g., on display 912 (FIG. 15).

Referring back to the workflow of FIG. 19, process 504 includes identifying, on the pre-operative image (FIG. 21), an angle α corresponding to a native posterior tibial slope angle of the patient. Process 504 may include a number of subprocesses 506, 508 510, and 512.

Subprocess 506 includes identifying an anatomical or mechanical axis 212 of the tibia 200.

Subprocess 508 includes creating a first reference line 210 perpendicular to the mechanical axis 212.

Subprocesses 506, 508 may be performed automatically by the surgical planning computer 910, while in others the identifying may be performed manually by the user, e.g., via visual identification of the respective axis/line on the image displayed on display 912, and user input provided to surgical planning computer 910 via a user interface.

Subprocess 510 includes creating or drawing a second reference line 214 on the 2D image, aligned with the patient's native PTS. Subprocess 510 may be performed relative to the medial or lateral side of the tibia, although in certain embodiments the lateral side may provide greater ease of visualization as compared to the medial side. Subprocess 510 may be performed manually by a user, e.g., via visual identification of the native posterior tibial slope on the image displayed on display 912, and user input provided to surgical planning computer 910 via a user interface to identify the native posterior tibial slope.

Subprocess 512 includes measuring the angle α between the first reference line 210 and the second reference line 214. The angle α corresponds to the native posterior tibial slope angle. Subprocess 512 may be performed automatically by the surgical planning computer 910.

Process 514 includes planning a tibial cut plane based on the posterior tibial slope angle α. In particular, the tibial cut plane is planned such that, when the tibia 200 is resected along the planned tibial cut plane, and a tibial tray is inserted onto a prepared proximal surface of the tibia, the resulting posterior slope of the tibial components (e.g., tibial tray) provides a posterior tibial slope corresponding to, e.g., substantially replicating that of the native posterior tibial slope of the patient 214.

Process 514 may include entering the angle α corresponding to the native tibial slope angle into a surgical computer platform such as, e.g., surgical planning computer 910, as a patient-specific parameter for planning the TKA procedure. The surgical planning computer 910 may be adapted to use the measured angle α as an input together with other parameters to pre-operatively generate a surgical plan, including a planned tibial cut plane, to provide post-operative kinematic alignment of the knee.

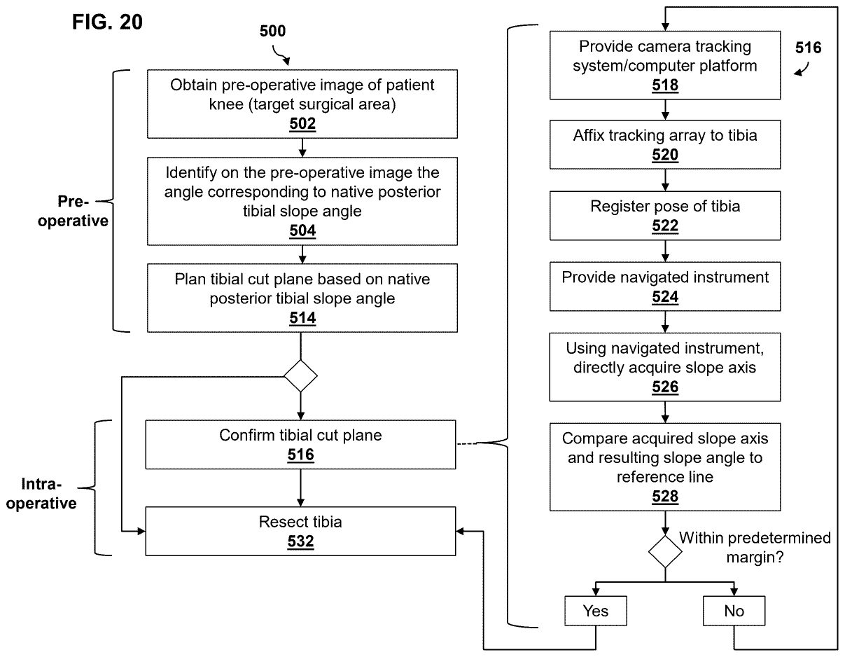

Referring to FIGS. 19-20, in certain embodiments, workflow 500 may additionally include an optional intra-operative process 516 to confirm the pre-operatively determined surgical plan, including the planned tibial cut plane. Process 516 may include sub-processes 518, 520, 522, 524, 526, and 528.

Subprocess 518 provides a computer platform such as, e.g., computer platform 900 including a surgical planning computer 910 (FIG. 15), and a camera tracking system such as, e.g., camera tracking system 6 (FIG. 11) as described herein. The camera tracking system is adapted to track a pose of one or more tracking arrays such as, e.g., DRA 52 (FIGS. 12-13), and is in communication with the computer platform as described above.

Subprocess 520 includes making an incision in the patient's leg, and affixing a first tracking array such as, e.g., DRA 52 (FIGS. 12-13), to the relevant tibia of the patient. The first tracking array comprises a plurality of reference elements adapted for tracking by the camera tracking system, as described herein.

Subprocess 522 includes registering a pose of the tibia 200 to the computer platform, as described herein. The computer platform may be adapted to automatically extract certain axes and landmarks such as, e.g., the mechanical axis 212 of the tibia 200, and the first reference line 210, which is perpendicular to the mechanical axis 212 as discussed above.

Subprocess 524 includes providing a navigated instrument 70 including a second tracking array such as a DRA 52 (FIG. 25), having a plurality of reference elements disposed thereon and adapted for tracking by the camera tracking system. The navigated instrument 70 may be, e.g., a navigated stylus, a pose of which may also be registered to the computer platform.

Subprocess 526, illustrated in FIG. 25, includes using the navigated instrument to directly acquire an axis corresponding to a native PTS 214 (FIG. 21) of the plateau 204 of the tibia 200, including aligning a shaft of the navigated instrument 70 with the native PTS (represented by line 214); and capturing an axis of the native PTS. The capturing may be performed by the computer platform in response to a user input, e.g., using a foot pedal or a user interface on a display. This provides a secondary or confirmatory process for assessing the patient's native posterior tibial slope.

Subprocess 528 includes comparing the acquired slope axis of subprocess 526, and the resulting slope angle between the acquired slope axis and the reference line 210 (which is in turn based on the mechanical axis 212), with the pre-operatively identified reference line 214 and measured angle α (process 504, including subprocesses 506-512). The slope axis angle as acquired in subprocess 526 may be deemed to confirm or be consistent with the pre-operative determination and corresponding surgical plan if it is within a predetermined margin. The slope axis as acquired in subprocess 526 may be deemed to fail to confirm, to conflict with, or be inconsistent withs the pre-operative determination and corresponding surgical plan if it is outside the predetermined margin. The predetermined margin may be, e.g., a margin of 1%, 2%, 5%, or similar.

When the slope axis as acquired in subprocess 526 confirms the pre-operative determination of PTS and corresponding surgical plan, the TKA procedure may proceed as pre-operatively planned. When the slope axis as acquired in subprocess 526 fails to confirm the pre-operative determination of PTS and corresponding surgical plan, the surgical plan may be revised prior proceeding with the TKA procedure. Revision of the surgical plan may include, e.g., repeating processes 504 and/or 516 as needed. A further process may include projecting the acquired native posterior tibial slope axis on a sagittal plane relative to the patient for use as a reference axis; and visually displaying an acquired native posterior tibial slope axis in an intra-operative implant planning view.

Following planning of the tibial cut plane in process 514, and optionally following confirmation of the planned tibial cut plane in process 516, the TKA procedure may be performed, including process 532 of resecting the tibia in accordance with the surgical plan.

The foregoing optional confirmatory process 516 is substantially analogous to the process described herein below relative to workflow 700 (FIG. 24), in which direct acquisition of the axis corresponding to native PTS is used as a primary method of determining PTS. Alternatively or additionally, a process such as described herein below relative to workflow 600 (FIG. 22) may be deployed as a confirmatory process in workflow 500 following tibial cut plane planning process 514, in accordance with embodiments of the disclosure.

Additional processes may be performed in addition to workflows 400 (FIGS. 16-17), 500 (FIGS. 19-20) to plan for the adjustment of other parameters, as described above with respect to FIGS. 6-8, e.g., varus/valgus rotation of the femur and tibia, and internal/external rotation of the femur. Collectively, the adjustments described relative to FIGS. 6-8 and the planning and adjustment of the PTS as described in workflows 400, 500 facilitate the pre-operative planning, optional intra-operative parameter confirmation, and surgical execution of a TKA procedure to provide a patient with kinematic alignment upon completion of the procedure.

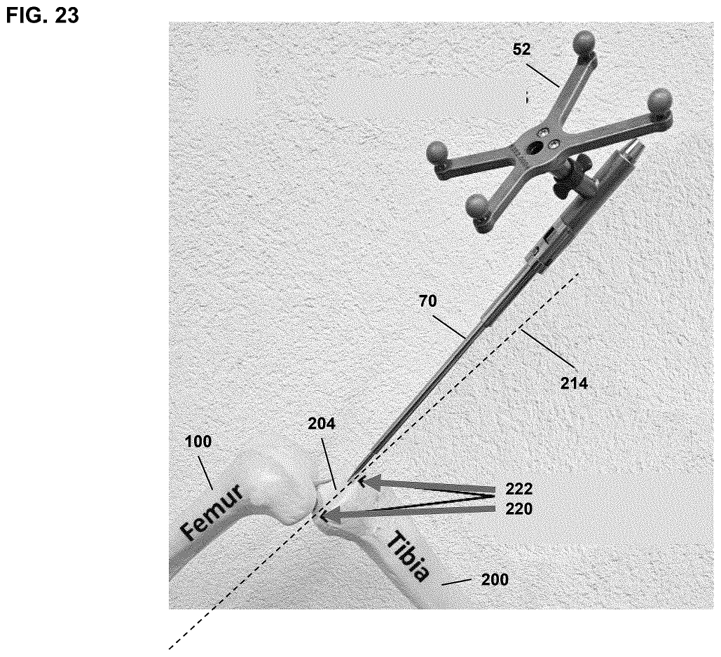

According to another embodiment, described with reference to FIGS. 22-23, a process is provided for performing a KA-TKA procedure on a patient, using an imageless workflow 600 (FIG. 22) for determining the patient's native PTS (FIG. 23) in the affected knee based on intra-operative point acquisition.

Workflow 600 includes process 602, which provides a computer platform such as, e.g., computer platform 900 including a surgical planning computer 910 (FIG. 15), and a camera tracking system such as, e.g., camera tracking system 6 (FIG. 11) as described herein. The camera tracking system is adapted to track a pose of one or more tracking arrays such as, e.g., DRA 52 (FIGS. 12-13), and is in communication with the computer platform as described above. Process 602 further includes providing a first tracking array 52, and a navigated instrument 70 (FIG. 23), e.g., a navigated stylus, comprising a second tracking array 52 having a plurality of reference elements thereon and adapted for tracking by the camera tracking system 6.

Process 604 includes making an incision in the patient's leg, and affixing a first tracking array such as, e.g., DRA 52 to the target tibia 200 of the patient. The first tracking array comprises a plurality of reference elements adapted for tracking by the camera tracking system, as described herein. Additional tracking arrays may be affixed to additional bones that are visible to the tracking camera such as, e.g., the femur 100.

Process 606 includes using the camera tracking system 6 and the first tracking array 52 to register a pose of the tibia 200 to the computer platform as described in, e.g., US Patent Application Publication No. US 2024/0020840 A1, previously incorporated by reference. Once registered, the pose of the tibia 200 may be displayed to the user in real time on a display device (such as, e.g., display 912 of FIG. 15) in communication with the computer platform, thus providing a visual representation of the tibia 200. The pose(s) of other bone(s) to which tracking arrays have been affixed may also be registered to the computer platform in an analogous manner.

Process 608 includes creating a reference line on the visual representation of the tibia 200, wherein the reference line corresponds to a native PTS of the patient. Process 608 may include subprocesses 610, 612, and 614.

As illustrated in FIG. 23, subprocess 610 includes acquiring, on a side of evaluation of the tibia, a first point 220 on a most prominent posterior aspect of a tibial plateau, and a second point 222 on a most prominent anterior aspect of the tibial plateau 204. In certain embodiments, the side of evaluation of the tibia 200 may be the medial side or the lateral side. The first and second points 220, 222 may be acquired using the navigated instrument 70, e.g., a navigated stylus, by contacting or palpating each of the first and second points 220, 222 with the tip of the navigated instrument 70. The camera tracking system captures the pose of the navigated instrument 70 as it individually contacts each of the points 220, 222 to be captured. The camera tracking system may thus capture the location of the distal tip of the navigated instrument 70 at the time of data acquisition, and store that location as a data point. The acquisition of each data point may be automatic or it may be user-actuated. For example, the user may instruct the computer platform to capture each data point, e.g., using any of various functionalities provided by the system such as, e.g., a foot pedal, a control on the stylus, a user interface on a touch screen display, or other device.

Subprocess 612 includes establishing a reference line 214 between the first (posterior) point 220 and the second (anterior) point 222 on the visual representation of the tibia 200. The visual representation of the tibia may be displayed, e.g., on display 912.

Subprocess 614 includes projecting the reference line 214 onto a sagittal plane relative to the patient. The native slope of the tibial plateau 204 may then be computed from the projected reference line 214. The native PTS may further be displayed, e.g. on the display device, in an intra-operative implant planning view.

With the reference line 214 thus created, process 616 includes intra-operatively planning a tibial cut plane based on a position or pose of the reference line 214. In particular, the tibial cut plane is planned such that, when the tibia 200 is resected along the planned tibial cut plane, and a tibial tray is inserted onto a prepared proximal surface of the tibia, the resulting posterior slope of the tibial components (e.g., tibial tray) provides a posterior tibial slope corresponding to, e.g., substantially replicating that of the native posterior tibial slope of the patient 214. The TKA procedure may then be performed according to the surgical plan, including resecting the tibia at process 632 in accordance with the planned tibial cut plane, based on the native PTS as determined in process 608.

According to a further embodiment, described with reference to FIGS. 24-25, a process is provided for performing a KA-TKA procedure on a patient, using an imageless workflow 700 (FIG. 24) for determining the patient's native posterior tibial slope (FIG. 25) in the affected knee based on direct acquisition of the posterior slope axis.

Workflow 700 includes process 702, which provides a computer platform such as, e.g., computer platform 900 including a surgical planning computer 910 (FIG. 15), and a camera tracking system such as, e.g., camera tracking system 6 (FIG. 11) as described herein. The camera tracking system is adapted to track a pose of one or more tracking arrays such as, e.g., DRA 52 (FIGS. 12-13), and is in communication with the computer platform as described above. Process 602 further includes providing a first tracking array 52, and a navigated instrument 70 (FIG. 23), e.g., a navigated stylus or a navigated tibial wall hook instrument, comprising a second tracking array 52 having a plurality of reference elements thereon and adapted for tracking by the camera tracking system 6.

Process 704 includes making an incision in the patient's leg, and affixing a first tracking array such as, e.g., DRA 52 to the target tibia 200 of the patient. The first tracking array comprises a plurality of reference elements adapted for tracking by the camera tracking system, as described herein. Additional tracking arrays may be affixed to additional bones that are visible to the tracking camera such as, e.g., the femur 100.

Process 706 includes using the camera tracking system 6 and the first tracking array 52 to register a pose of the tibia 200 to the computer platform as described in, e.g., US Patent Application Publication No. US 2024/0020840 A1, previously incorporated by reference. Once registered, the pose of the tibia 200 may be displayed to the user in real time on a display device (such as, e.g., display 912 of FIG. 15) in communication with the computer platform, thus providing a visual representation of the tibia 200. The pose(s) of other bone(s) to which tracking arrays have been affixed may also be registered to the computer platform in an analogous manner.

Process 708 includes using the navigated instrument 70 to directly acquire an axis corresponding to a native posterior tibial slope of the tibia. Process 708 includes subprocesses 710, 712, and 714.

As illustrated in FIG. 25, subprocess 710 includes aligning a shaft of the navigated instrument 70 with the native posterior slope of the tibial plateau 204 on either the medial side or the lateral side, such that the shaft of the navigated instrument 70 is tangent to the posterior tibial plateau 204.

Subprocess 712 includes capturing an axis 214 of the native PTS when the shaft of the navigated instrument is tangent to the posterior tibial plateau 204. The camera tracking system captures the pose of the navigated instrument 70 when the shaft of the navigated instrument 70 is positioned tangent to the posterior tibial plateau 204 as described above, and the computer platform stores the captured location. The acquisition of the axis may be automatic or may be user-actuated, e.g., in response to user input. For example, the user may instruct the computer platform to capture the axis 214 using any of various functionalities provided by the system such as, e.g., a foot pedal such as, e.g., a foot pedal 880 (FIG. 14) in communication with the computer platform, a control on the stylus, a user interface displayed on a display device such as, e.g., display 824 or tablet 890 in communication with the computer platform 800, or another device.

Subprocess 714 includes projecting the acquired axis 214 onto a sagittal plane relative to the patient. The native posterior slope of the tibial plateau 204 may then be computed from the projected axis. The native posterior tibial slope may further be displayed, e.g. on the display device, in an intra-operative implant planning view.

Process 716 includes intra-operatively planning a tibial cut plane based on a pose of the acquired axis 214. In particular, the tibial cut plane is planned such that, when the tibia 200 is resected along the planned tibial cut plane, and a tibial tray is inserted onto a prepared proximal surface of the tibia, the resulting posterior slope of the tibial components (e.g., tibial tray) provides a posterior tibial slope corresponding to, e.g., substantially replicating that of the native posterior tibial slope of the patient 214. In certain embodiments, the intra-operative planning further includes selecting a tibial insert component for insertion based on the position of the axis.

The TKA procedure may then be performed according to the surgical plan, including resecting the tibia at process 732 in accordance with the planned tibial cut plane, based on the axis corresponding to the patient's native PTS, as acquired in process 708.

Additional processes may be performed in addition to workflows 600 (FIG. 22), 700 (FIG. 24) to plan for the adjustment of other parameters, as described above with respect to FIGS. 6-8, e.g., varus/valgus rotation of the femur and tibia, and internal/external rotation of the femur, and tibial insert component selection. Collectively, the adjustments described relative to FIGS. 6-8 and the planning and adjustment of the PTS as described in workflows 600, 700 facilitate the intra-operative planning and surgical execution of a TKA procedure to provide a patient with kinematic alignment upon completion of the procedure.

In the above description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected,” “coupled,” “responsive,” or variants thereof to another element, it can be directly connected, coupled, or responsive to the other element or intervening elements may be present. In contrast, when an element is referred to as being “directly connected,” “directly coupled,” “directly responsive,” or variants thereof to another element, there are no intervening elements present. Like numbers refer to like elements throughout. Furthermore, “coupled,” “connected,” “responsive,” or variants thereof as used herein may include wirelessly coupled, connected, or responsive. As used herein, the singular forms “a,” “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise,” “comprising,” “comprises,” “include,” “including,” “includes,” “have,” “has,” “having,” or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

1. A method for planning a total knee arthroplasty (TKA) procedure on a patient, comprising:
obtaining a pre-operative image of a knee of the patient;
identifying, on the pre-operative image, a reference line corresponding to a native posterior tibial slope of the patient; and
planning a tibial cut plane based on the reference line
wherein the pre-operative image comprises a 3D model of a tibia of the patient generated from a CT scan or an MRI scan,
wherein identifying the reference line comprises:
extracting from the 3D bone model a pre-selected proximal plateau point at one or both of a medial side or a lateral side of the tibia of the patient;
creating a measurement plane parallel to a plane running through the patient;
translating the measurement plane to the pre-selected proximal plateau point corresponding to a side of evaluation; and
extracting a first most proximal point and a second most proximal point of the 3D bone model intersecting the measurement plane.

2. The method of claim 1, wherein the tibial cut plane is planned such that,
when a tibia of the patient is cut along the tibial cut plane, and
a tibial tray is inserted onto a prepared proximal surface of the tibia,
the tibial tray provides a posterior tibial slope corresponding to the native posterior tibial slope of the patient.

3. (canceled)

4. (canceled)

5. The method of claim 1, wherein the pre-selected proximal plateau point is extracted on the medial side of the tibia.

6. The method of claim 1, wherein steps of:
extracting the pre-selected proximal plateau point, and
extracting first most proximal point and the second most proximal point, are performed automatically using a surgical computer platform.

7. The method of claim 1, further comprising drawing the reference line on the pre-operative image between the first most proximal point and the second most proximal point, wherein the reference line corresponds to the native posterior tibial slope.

8. The method of claim 1, further comprising intra-operatively confirming a position of the reference line corresponding to the native tibial slope by:
providing a camera tracking system adapted to track a pose of one or more tracking arrays, and a computer platform in communication with the camera tracking system;
affixing a first tracking array to a tibia of the patient, wherein the first tracking array comprises a plurality of reference elements adapted for tracking by the camera tracking system;
registering a pose of the tibia to the computer platform;
providing a navigated instrument comprising a second tracking array having a plurality of reference elements thereon and adapted for tracking by the camera tracking system;
using the navigated instrument, directly acquiring an axis corresponding to a native posterior tibial slope of the tibia; and
confirming the position of the reference line as identified pre-operatively.

9. The method of claim 8, wherein the directly acquiring comprises:
aligning a shaft of the navigated instrument with the native posterior tibial slope; and
capturing an axis of the native posterior tibial slope in response to a user input.

10. The method of claim 8, further comprising:
projecting the acquired native posterior tibial slope axis on a sagittal plane relative to the patient for use as a reference axis; and
visually displaying an acquired native posterior tibial slope axis in an intra-operative implant planning view.

11. (canceled)

12. (canceled)

13. (canceled)

14. (canceled)

15. (canceled)

16. (canceled)

17. (canceled)

18. (canceled)

19. (canceled)

20. (canceled)
